(19) United States
(12) Patent Application Publication
(10) Pub. No.: US 2015/0245810 A1
(43) Pub. Date: Sep. 3, 2015

(54) ULTRASOUND GEL DISPENSING AND PROBE SANITIZING STATION

(71) Applicant: dBMEDx Inc., Littleton, CO (US)

(72) Inventors: David B. Shine, Littleton, CO (US); William L. Barnard, Maple Valley, WA (US); William Quirk, Littleton, CO (US); William A. Marty, Seattle, WA (US)

(73) Assignee: dBMEDx Inc., Littleton, CO (US)

(21) Appl. No.: 14/195,731

(22) Filed: Mar. 3, 2014

(51) Int. Cl. A61B 8/00; B65D 83/00

(52) U.S. Cl. CPC A61B 8/4405; B65D 83/0083

(57) ABSTRACT

A cart includes a power supply, a display unit, a holder having a primary coil positioned with respect to a compartment to be proximate a secondary coil of a handheld ultrasound probe when it is positioned in the compartment, a charging circuit that provides an alternating current to inductively couple the primary and the secondary coils, light sources operable to provide at least ultraviolet light, and a drive circuit electrically coupled to drive the light sources. The cart may include a dispenser with a container that can hold an ultrasound coupling medium, and a dispensing mechanism having a scoop with an elongated recess that is rotatably mounted with respect to the container to move between a first position in which the elongated recess is in fluid communication with the interior of the container and a second position in which the elongated recess is exposed to an exterior of the dispenser.

### BACKGROUND

1. Technical Field

This disclosure generally relates to devices that may be used with ultrasound equipment, and more particularly to devices that may be used with handheld ultrasound probes.

2. Description of the Related Art

Medical ultrasound requires the use of an ultrasound coupling medium to permit sonic energy to be efficiently transmitted into and received from the body of a patient. Typically, the coupling medium is in the form of a gel comprised primarily of water and having a consistency ranging from a tough gelatin to a runny paste. Generally, the more viscous the gel the better the ultrasound coupling properties; the vast majority of ultrasound exams are performed with a relatively viscous gel.

In a typical hospital setting, an ultrasound coupling medium is in the form of a water-based gel that is stored in a bottle formed from a pliable plastic material that an operator can squeeze to force the coupling medium out of a nozzle and onto the probe of an ultrasound device, the skin a patient, or both. Because the gel is generally stored at a room temperature that is over 25° F. cooler than the temperature of the patient's body, the patient may experience mild discomfort when the gel is initially applied. This is exacerbated by the very same properties that are useful for sonic coupling, because the water-based gel spreads out quickly and acts as a good thermal coupler. To provide a more comfortable experience to patients, ultrasound technicians may use conventional devices to electrically warm disposable bottles of ultrasound coupling media.

Because disposable bottles of gel for medical ultrasound contain only a limited amount of the gel, they tend to run out of the gel quickly. Additionally, such bottles become increasingly difficult to manipulate with use. For example, it may be difficult to squeeze out that last remaining amount of gel from such a bottle, which may result in an operator violently shaking the bottle in an upside-down position to attempt to move the remaining gel to an end of the bottle at which a dispensing nozzle is located. In addition, two-handed operation is typically required with such gel bottles regardless of whether the gel is applied to an ultrasound probe or to a patient's skin. For example, the operator may hold the ultrasound probe in one hand while manipulating a bottle containing a gel with the other hand to apply the gel to the ultrasound probe or the patient's skin.

Another issue associated with the use of medical ultrasound is cleaning, sanitizing, and sterilization of ultrasound probes. Ultrasound procedures typically require a liquid interface and skin contact, which may contaminate an ultrasound probe with pathogens from patients. To avoid cross contamination from one patient to another, a disposable plastic sheath or condom may be placed over the ultrasound probe. This takes extra time to deploy and provides an additional barrier that may obstruct and degrade the quality of ultrasound imaging. When a sheath is used, an ultrasound coupling gel typically must be used inside the sheath and on the outside of the sheath to provide adequate coupling, which is messy and increases the amount of gel that gets onto the ultrasound probe. In addition, the gel itself is typically not sterile and is dispensed from a multi-use bottle. Disposable sheaths are rarely used in practice because many operators consider them to be inconvenient. Moreover, because many operators do not use disposable gloves when performing ultrasound exams, the chances of contamination of the ultrasound probe increases.

### BRIEF SUMMARY

This disclosure describes apparatuses, dispensers, and carts that overcome one or more of the problems identified above. For example, the present disclosure describes a portable station in the form of a cart with an apparatus that can sanitize an ultrasound probe while it is being charged and a gel dispenser that can be operated with one hand to improve an operator's experience and that can warm the gel to enhance a patient's experience.

An apparatus for use with a handheld ultrasound probe, the handheld ultrasound probe which includes a power storage device, may be summarized as including: a holder that forms a compartment, the compartment sized and dimensioned to receive at least a portion of the handheld ultrasound probe therein; a primary coil positioned with respect to the compartment to be proximate a secondary coil of the handheld ultrasound probe when the handheld ultrasound probe is positioned in the compartment; a charging circuit electrically coupled to the primary coil to selectively provide an alternating current therethrough of sufficient strength to inductively couple the primary and the secondary coils when the handheld ultrasound probe is positioned in the compartment; a plurality of light sources selectively operable to provide light at least from an ultraviolet band of an electromagnetic spectrum in at least part of the compartment to substantially illuminate the handheld ultrasound probe when the handheld ultrasound probe is positioned in the compartment; and a drive circuit electrically coupled to drive the light sources.

The apparatus may further include a cover, wherein at least one of the cover and the compartment is selectively movable between at least an open position in which the compartment is open to an exterior of the holder and a closed position in which the compartment is closed to the exterior of the holder. The drive circuit may drive the light sources to output ultraviolet light only when the cover or the compartment is in the closed position. At least a first portion of the holder may include at least one material that is transmissive of ultraviolet light. The first portion of the holder may be transmissive of visible light, and at least a second portion of the holder may be formed from one or more materials that is nontransmissive of ultraviolet light. The light sources may include at least one ultraviolet light emitting diode. The apparatus may further include at least one adhesive that is transmissive of ultraviolet light and that secures the light sources to the holder. At least one of the light sources may be positioned adjacent a center portion of the primary coil and one or more one of the light sources may be positioned adjacent an outer periphery of the primary coil. The primary coil may induce an electromagnetic field in the secondary coil of the ultrasound probe while the light sources output light.

A dispenser to dispense ultrasound coupling media may be summarized as including a container having an interior that in use holds a supply of an ultrasound coupling medium; and a dispensing mechanism having a scoop including at least an elongated recess, the elongated recess sized and dimensioned to hold a quantity of the ultrasound coupling medium, the scoop rotatably mounted with respect to the container to selectively move between at least a first position in which the elongated recess is in fluid communication with the interior of the container and to at least a second position in which the elongated recess is exposed to an exterior of the dispenser to dispense the quantity of the ultrasound coupling medium.

The scoop may be an elongated cylindrical member with the elongated recess formed in a peripheral surface thereof. The scoop may be an elongated cylindrical member with the elongated recess having an arcuate profile. The dispensing mechanism may include a plunger disposed in the container, the plunger movable to urge at least some of the ultrasound coupling medium into the elongated recess, and the dispensing mechanism may also include means for biasing the plunger toward the scoop. The dispensing mechanism may include an actuator coupled to the scoop, wherein movement of the at least part of the actuator causes the scoop to rotate at least between the first and second positions. The actuator may include a first plurality of teeth, the scoop may be coupled to a second plurality of teeth that engage the first plurality of teeth, and movement of the first plurality of teeth may cause the scoop to rotate. The dispenser may further include a heater in thermal communication with the container or the interior of the container; and the container may include at least one thermal insulator at least partially enclosing the interior thereof. The dispenser may further include a hanger to detachably couple the dispenser to a pole. The ultrasound coupling media may be a gel contained in a bag that is removably received in the container.

A cart may be summarized as including: a frame; a plurality of wheels rotatably mounted to the frame; a power supply carried by the frame; a display unit carried by the frame, electrically coupled to the power supply; an ultrasound probe holder that forms a compartment, the compartment sized and dimensioned to receive at least a portion of the handheld ultrasound probe therein; a primary coil positioned with respect to the compartment to be proximate a secondary coil of the handheld ultrasound probe when the handheld ultrasound probe is positioned in the compartment; a charging circuit electrically coupled to the power supply and to the primary coil to selectively provide an alternating current therethrough of sufficient strength to inductively couple the primary and the secondary coils when the handheld ultrasound probe is positioned in the compartment; a plurality of light sources selectively operable to provide light at least from an ultraviolet band of an electromagnetic spectrum in at least part of the compartment to substantially illuminate the handheld ultrasound probe when the handheld ultrasound probe is positioned in the compartment; and a drive circuit electrically coupled to drive the light sources.

The display unit may display images based on data wirelessly received from the ultrasound probe. The ultrasound probe holder may be formed from at least one material that is transmissive of ultraviolet light. The primary coil may induce an electromagnetic field in the secondary coil of the ultrasound probe while the light sources output light. The cart may further include a cover, wherein at least one of the cover and the compartment is selectively movable between at least an open position in which the compartment is open to an exterior of the holder and a closed position in which the compartment is closed to the exterior of the holder. The drive circuit may drive the light sources to output ultraviolet light only when the cover or the compartment is in the closed position. At least a first portion of the holder may include at least one material that is transmissive of ultraviolet light. The power supply may include at least one battery. The cart may further include a dispenser including a container having an interior that in use holds a supply of an ultrasound coupling medium; and a dispensing mechanism having a scoop including at least an elongated recess, the elongated recess sized and dimensioned to hold a quantity of the ultrasound coupling medium, the scoop rotatably mounted with respect to the container to selectively move between at least a first position in which the elongated recess is in fluid communication with the interior of the container and to at least a second position in which the elongated recess is exposed to an exterior of the dispenser to dispense the quantity of the ultrasound coupling medium. The cart may further include a heater in thermal communication with the container or the interior of the container; and the container may include at least one thermal insulator at least partially enclosing the interior thereof. The cart may further include a hanger to detachably couple the dispenser to the frame.

### BRIEF DESCRIPTION OF THE SEVERAL VIEWS OF THE DRAWINGS

FIG. 1 is a top view of a charging and sanitizing apparatus, according to one illustrated embodiment.

FIG. 2 is a front view of the charging and sanitizing apparatus shown in FIG. 1.

FIG. 3 is a top view of a portion of the charging and sanitizing apparatus shown in FIG. 1.

FIG. 4 is a side view of the portion of the charging and sanitizing apparatus shown in FIG. 3 with a part of a side portion thereof cut away.

FIG. 5 is a block diagram of a charging and sanitizing apparatus, according to one illustrated embodiment.

FIG. 6 is a front view of a dispenser, according to one illustrated embodiment.

FIG. 7 is a side view of the dispenser shown in FIG. 6.

FIG. 8 is a cross-sectional view of the dispenser shown in FIG. 7.

FIGS. 9A-9C are cross-sectional views of a first portion of the dispenser shown in FIG. 8, at various stages of operation.

FIGS. 10A-100 are cross-sectional views of a second portion of the dispenser shown in FIG. 8, at various stages of operation.

FIG. 11 is a cross-sectional view of a portion of a dispenser, according to one illustrated embodiment.

FIG. 12 is a block diagram of a dispenser, according to one illustrated embodiment.

FIG. 13 is a front view of a cart, according to one illustrated embodiment.

FIG. 14 is a cross-sectional view of the cart shown in FIG. 13.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with power supplies, inductive chargers, motors, electric heaters, light sources, and drive circuits for same have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one illustrated embodiment” or “an embodiment” means that a particular feature, structure or characteristic described in connection with the embodiment is included in at least one illustrated embodiment. Thus, the appearances of the phrases “in one illustrated embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

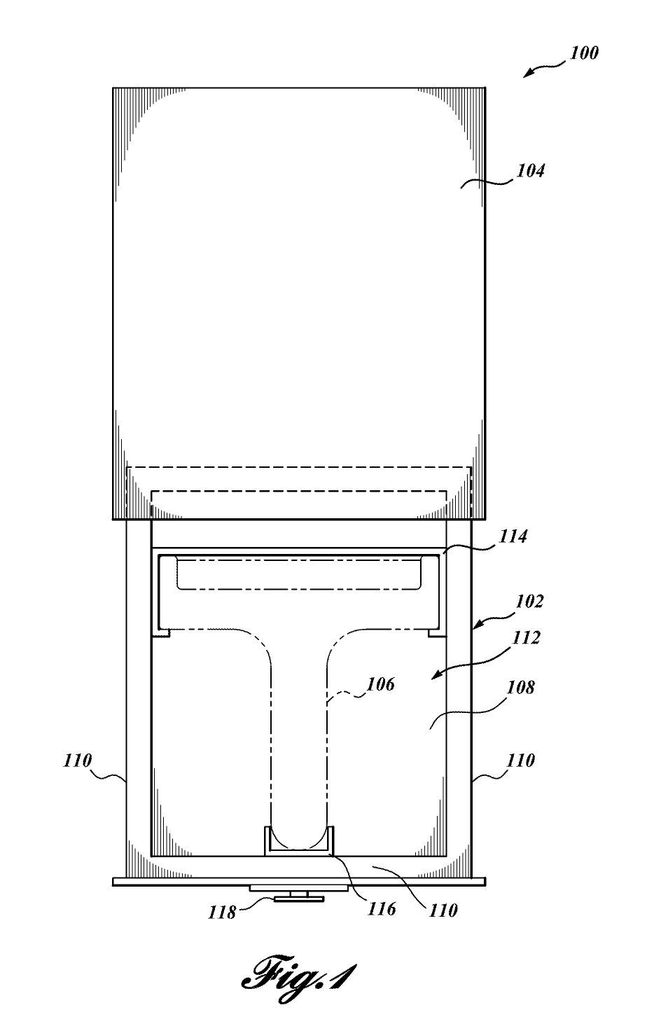

FIGS. 1-4 show an ultrasound probe charging and sanitizing apparatus 100, according to one illustrated embodiment. The apparatus 100 includes a holder 102 and a cover 104. The holder 102 includes a base portion 108 and four side portions 110 disposed about a periphery of the base portion 108. The base portion 108 and the side portions 110 of the holder 102 define a compartment 112 that is sized and dimensioned to receive at least a portion of a handheld ultrasound probe 106 therein. In one embodiment, the handheld ultrasound probe 106 is an automated bladder volume measurement device of a Benchmark Bladder System™ available from dBMEDx, Inc.

A first guide 114 and a second guide 116 are disposed in the compartment 112 of the holder 102. As will be described in greater detail below, the first guide 114 and the second guide 116 enable the handheld ultrasound probe 106 to be precisely positioned within the compartment 112. In one embodiment, the first guide 114 and the second guide 116 are formed from Corning® Gorilla® Glass, which is transmissive of ultraviolet (UV) light. In one embodiment, the first guide 114 and the second guide 116 are formed from a clear, UV transmissive, and scratch-resistant glass and/or plastic material.

The holder 102 may function like a drawer. For example, the holder 102 may be mounted to rails (not illustrated) that facilitate movement of the holder 102 with respect to the cover 104. A knob 118 may be attached to one of the side portions 110. An operator may pull on the knob 118 to move at least part of the holder 102 from under the cover 104 to at least partially expose the contents of the compartment 112 of the holder 102 to an exterior of the ultrasound probe charging and sanitizing apparatus 100, as shown in FIG. 1. In one embodiment, the cover 104 is pivotally attached to the holder 102 (e.g., using one or more hinges) such that an operator may lift the cover 104 to expose the contents of the compartment 112 of the holder 102 to an exterior of the ultrasound probe charging and sanitizing apparatus 100.

As best illustrated in FIG. 3, a plurality of light sources 120 is mounted with respect to the holder 102. For illustrative simplicity, only two of the light sources 120 are shown in FIG. 3. Additional light sources 120 (not shown) may be mounted to other portions of the first guide 114 and to portions of the second guide 116. Also, additional light sources 120 (not shown) may be mounted above and below the holder 102 so that substantially all of the outer surfaces of the handheld ultrasound probe 106 are sufficiently irradiated with UV light to sanitize those surfaces.

The light sources 120 are selectively operable to provide light at least from an ultraviolet band of an electromagnetic spectrum in at least part of the compartment 112 to substantially illuminate the handheld ultrasound probe 106 when the handheld ultrasound probe 106 is positioned in the compartment 112. The light sources 120 shown in FIG. 3 are mounted to the first guide 114 using an adhesive 122 that is transmissive of ultraviolet light. In one embodiment, at least some of the light sources 120 are UV light emitting diodes (LEDs) that have wide dispersing UV lens. In one embodiment, the light sources 120 are UV LEDs that produce wavelengths of UV light in a range from about 380 nanometers (nm) to about 395 nm.

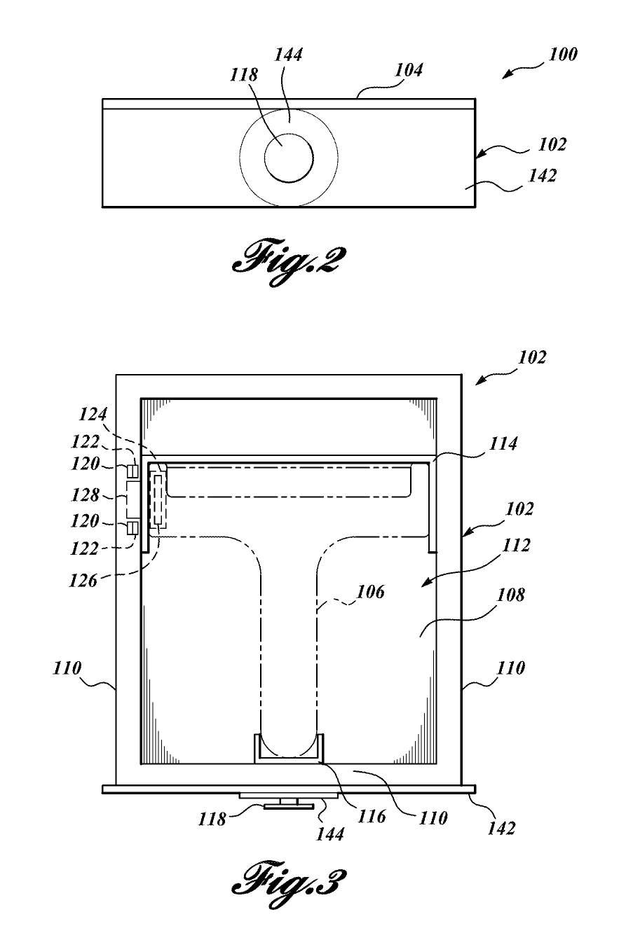

In one embodiment, the base portion 108, the side portions 110, and a front portion 142 of the holder 102 are made from a material that is transmissive of visible light, but is not transmissive of UV light. A plate 144 that is not transmissive of visible and UV light is disposed between the knob 118 and the front portion 142. The plate 144 prevents light sources 120 (not shown) that are adhered to the front of the second guide 116 and corresponding wires from being visible, for example, when the holder 102 is viewed from the front as shown in FIG. 2.

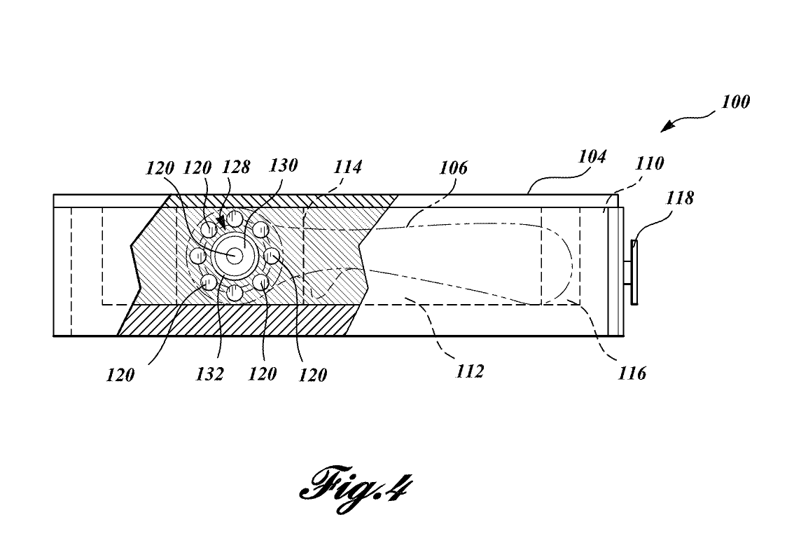

As best illustrated in FIG. 4, the coil unit 128 includes a primary coil 130 that is surrounded by a ferrous annular ring 132. The primary coil 130 may include a plurality of loops or coils of a copper wire. In the illustrated embodiment, one of the light sources 120 is positioned adjacent a center portion of the primary coil 130 and several of the light sources 120 are positioned adjacent an outer periphery of the primary coil 130.

Referring now to FIGS. 3 and 4, the handheld ultrasound probe 106 includes a power storage device 124 that is electrically coupled to a secondary coil 126. The primary coil 130 is positioned with respect to the compartment 112 to be proximate the secondary coil 126 of the handheld ultrasound probe 106 when the handheld ultrasound probe 106 is positioned in the compartment 112.

FIG. 5 schematically illustrates the charging and sanitizing apparatus 100 of FIGS. 1-4. The charging and sanitizing apparatus 100 includes a controller 134 that is electrically coupled to a charging circuit 136. The charging circuit 136 is electrically coupled to a power supply (not shown in FIG. 5) and to the primary coil 130 to selectively provide an alternating current therethrough of sufficient strength to inductively couple the primary coil 130 of the charging and sanitizing apparatus 100 and the secondary coil 126 of the handheld ultrasound probe 106. When the alternating current is inductively coupled to the primary coil 130 and the secondary coil 126, a current may flow from the secondary coil 130 to the power storage device 124, which charges the power storage device 124.

The controller 134 also is electrically coupled to a switch or sensor 138 and a drive circuit 140, which drives the light sources 120. In one embodiment, the sensor 138 provides a signal to the controller 134 when the compartment 112 is not exposed to an exterior of the holder 102, and does not provide the signal to the controller 134 when the compartment 112 is exposed to the exterior of the holder 102. For example, the compartment 112 is not exposed to the exterior of the holder 102 when the entire compartment 112 is in a closed position beneath the cover 104. So long as the sensor 138 provides the signal to the controller 134, the controller 134 provides a signal to the drive circuit 140, which enables the drive circuit 140 to drive the light sources 120. Accordingly, the drive circuit 140 may drive the light sources 120 to output UV light only when the compartment 112 (or the cover 104) is in a closed position.

In that connection, the cover 104 and the base portion 108 and the side portions 110 of the holder 102 are formed from at least one material that is not transmissive of the UV light output from the light sources 120. Because it can be useful to know whether the handheld ultrasound probe 106 is in the holder 102, in one embodiment, the cover 104 and the side portions 110 and the front portion 142 of the holder 102 are formed from one or more materials that are transmissive of visible light and that are not transmissive of UV light. For example, the cover 104 and the side portions 110 and the front portion 142 of the holder 102 may be formed from the Optivex™ UV Filter from the GrayGlass Company, which blocks 99% of light having wavelengths below 400 nm. Alternatively, the cover 104 and the side portions 110 and the front portion 142 of the holder 102 may be formed from a transparent glass or plastic on which a coating has been formed, wherein the coating blocks 99% of light having wavelengths below 400 nm.

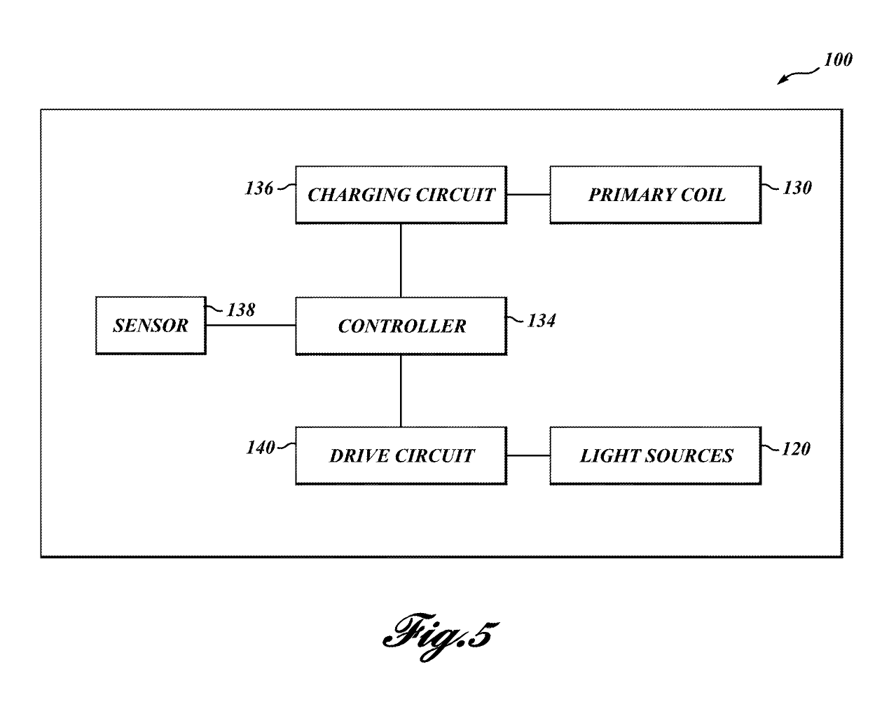

FIGS. 6-8 show a dispenser 200, according to one illustrated embodiment. The dispenser 200 includes a housing 202. As shown in FIG. 6, a portion of a remaining quantity indicator 204 extends from an upper portion of the housing 202 and portions of a first activation button 206a and a second activation button 206b extend from a lower portion of the housing 202.

As shown in FIG. 8, the dispenser 200 includes a container 208 having an interior that may contain a supply of an ultrasound coupling medium 210. A dispensing mechanism 212 dispenses the ultrasound coupling medium 210 from the container 208. The dispensing mechanism 212 includes a scoop 214 that is rotatably coupled to the container 208.

More particularly, the scoop 214 includes a first axle 216a that extends outwardly from a first end portion 218a of the scoop 214 through an aperture formed in a first side portion 220a of the container 208. The scoop 214 also includes a second axle 216b that extends outwardly from a second end portion 218b of the scoop 214 through an aperture formed in an opposing second side portion 220b of the container 208. A pair of gears 222 is coupled to the first axle 216a and the second axle 216b; however, only one of the gears 222 is shown in FIG. 8. In that connection, only a portion of the first activation button 206a is shown in FIG. 8 so that the gear 222 coupled to the first axle 216a can be seen in FIG. 8. The structure of the first activation button 206a is substantially the same as that of the second activation button 206b. When the gears 222 are rotated, the scoop 214 rotates about the first axle 216a and the second axle 216b.

The dispenser 200 also includes a plunger 224 that can move from the top of the container 208 toward the scoop 214. A weight 226 is disposed on the plunger 224. Gravity pulls the weight 226, and thus the plunger 224, toward the scoop 214. The weight 226 is coupled to the remaining quantity indicator 204. The weight 226 and the remaining quantity indicator 204 move downwardly as the dispensing mechanism 212 dispenses the ultrasound coupling medium 210 from the dispenser 200.

The remaining quantity indicator 204 includes a plurality of markings 204a, which may be printed on or embossed into the remaining quantity indicator 204, for example. The markings 204a are positioned on the remaining quantity indicator 204 to indicate the quantity of the ultrasound coupling medium 210 remaining in the container 208. For example, if 50% of the ultrasound coupling medium 210 that was originally contained in the container 208 has been dispensed from the dispenser 200, a marking 204a labeled “50%” may be visible just above the upper surface of the housing 202.

The dispenser 200 also may include a first heater 228a adjacent the first side portion 220a of the container 208 and a second heater 228b adjacent the second side portion 220b of the container 208. The first heater 228a and the second heater 228b are in thermal communication with the interior of the container 208. In one embodiment, the first heater 228a and the second heater 228b are resistance heaters. Additionally, the dispenser 200 may include a first thermal insulator portion 230a and a second thermal insulator portion 230b that at least partially enclose the interior of the container 208. The first thermal insulator portion 230a and the second thermal insulator portion 230b may keep the interior of the container 208 and thus the ultrasound coupling medium 210 warm after the first heater 228a and the second heater 228b have stopped producing heat.

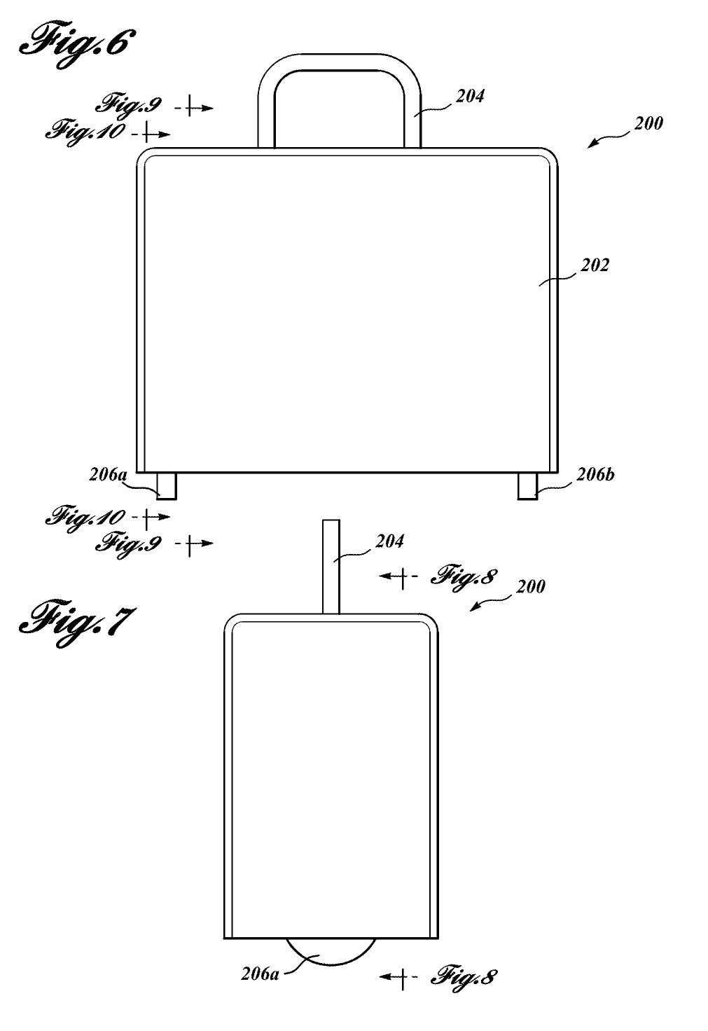

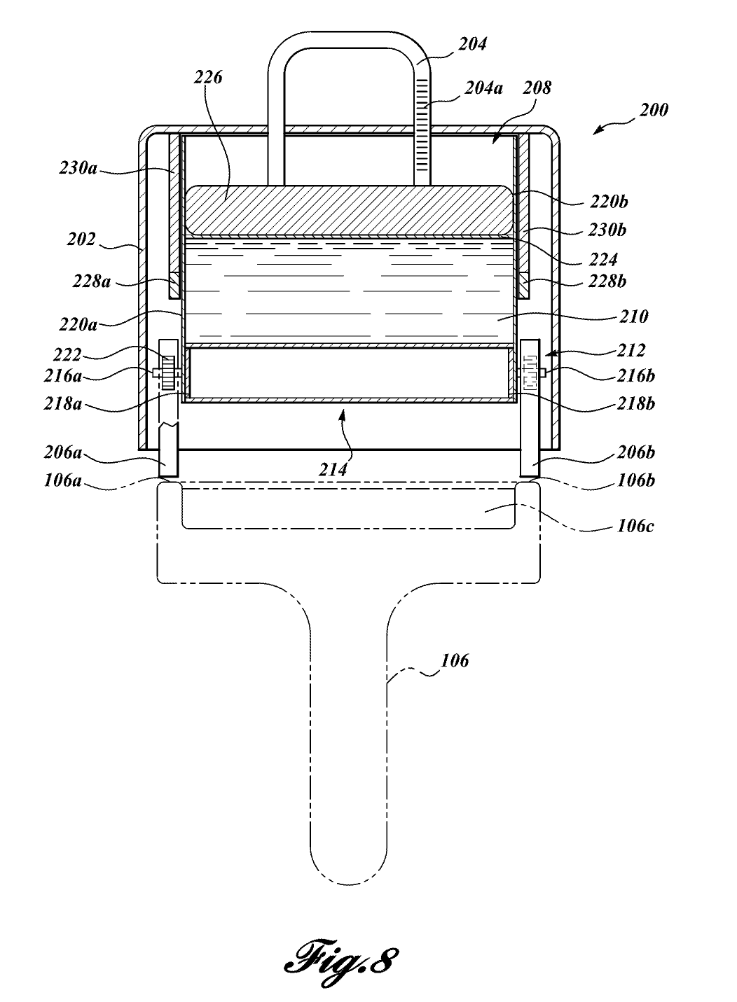

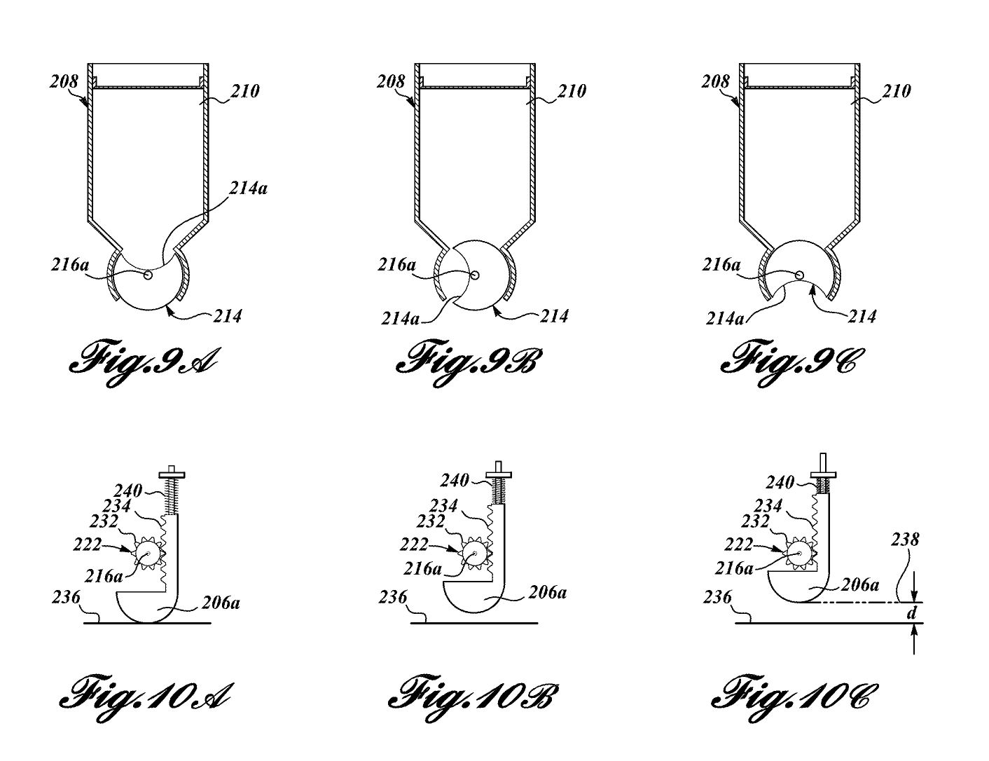

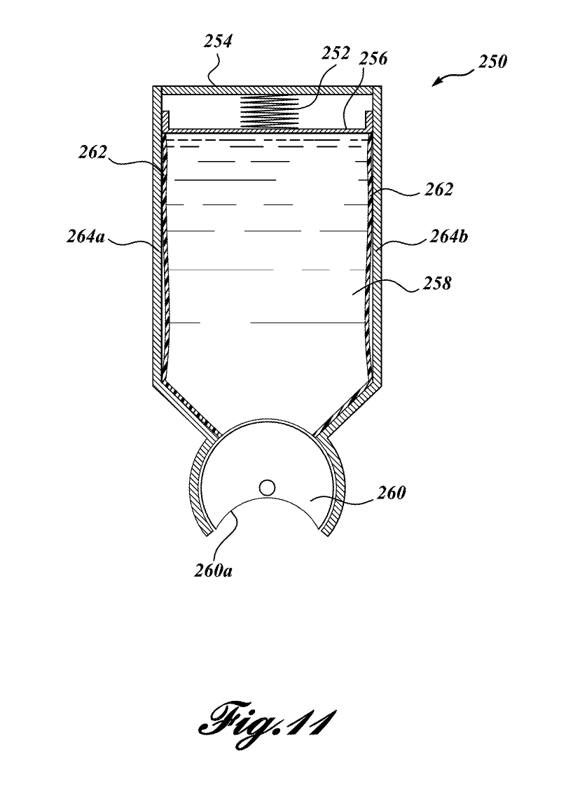

FIGS. 9A-9C show a first portion of the dispenser 200 showing the container 208 and the scoop 214 at various stages of operation. FIGS. 10A-10C show a second portion of the dispenser 200 showing the gear 222 and the first activation button 206a at the same stages of operation as in FIGS. 9A-9C, respectively.

Operation of the dispenser 200 will now be described with reference to FIGS. 8, 9A-9C, and 10A-100. FIGS. 9A and 10A correspond to default positions of the scoop 214 and the first activation button 206a, respectively. For example, FIGS. 9A and 10A correspond to a situation in which an operator holds the handheld ultrasound probe 106 such that a first portion 106a of the handheld ultrasound probe 106 is vertically aligned with the first activation button 206a and a second portion 106b of the handheld ultrasound probe 106 is vertically aligned with the second activation button 206b, as shown in FIG. 8.

As shown in FIGS. 8 and 9A-9C, the scoop 214 is an elongated cylindrical member with an elongated recess 214a having an arcuate profile. When the scoop 214 is in the default position shown in FIG. 9A, the elongated recess 214a is in fluid communication with the interior of the container 208. A reference marker 236 is shown in FIGS. 10A-10C to indicate the position of the bottom of the first activation button 206a when the first activation button 206a is in the default position.

FIGS. 9B and 10B correspond to intermediate positions of the scoop 214 and the first activation button 206a, respectively. For example, FIGS. 9B and 10B correspond to a situation in which the operator moves the handheld ultrasound probe 106 upwardly from the position shown in FIG. 8 such that the first portion 106a and the second portion 106b of the handheld ultrasound probe 106 contact the first activation button 206a and the second activation button 206b, respectively, causing the first activation button 206a and the second activation button 206b to move upwardly a short distance.

The gear 222 includes a plurality of teeth 232 that engage a plurality of teeth 234 formed on the first activation button 206a. Accordingly, when the first activation button 206a is moved upwardly, the gear 222 rotates counterclockwise, which cause the scoop 214 to rotate counterclockwise. When the first activation button 206a is moved upwardly, an upper portion of the first activation button 206a compresses a spring 240.

FIGS. 9C and 100 correspond to end positions of the scoop 214 and the first activation button 206a, respectively. FIGS. 9C and 100 correspond to a situation in which the operator moves the handheld ultrasound probe 106 upwardly from the position shown in FIG. 8 a predetermined distance such that the first activation button 206a and the second activation button 206b sufficiently actuate the gears 222 to cause the scoop 214 to rotate counterclockwise 180° so that the elongated recess 214a is exposed to an exterior of the container 208.

As shown in FIG. 8, a portion 106c of the handheld ultrasound probe 106 may be positioned directly below the scoop 214. For example, the portion 106c of the handheld ultrasound probe 106 contains ultrasound transducers. In one embodiment, the first activation button 206a and the second activation button 206b are disposed between a plurality of guides (not shown) that extend from the lower portion of the housing 202 to ensure that the handheld ultrasound probe 106 is in a predetermined position with respect to the scoop 214 (e.g., the position of the handheld ultrasound probe 106 shown in FIG. 8) when the handheld ultrasound probe 106 contacts the first activation button 206a and the second activation button 206b.

When the scoop 214 is in the position shown in FIG. 9C, the elongated recess 214a is exposed to an exterior of the dispenser 200 and substantially all of the ultrasound coupling medium 210 disposed in the elongated recess 214a, if any, falls onto the portion 106c of the handheld ultrasound probe 106. Because the elongated recess 214a has a predetermined volume, when the elongated recess 214a is filled with the ultrasound coupling medium 210, a predetermined quantity of the ultrasound coupling medium 210 falls onto the portion 106c of the handheld ultrasound probe 106 when the handheld ultrasound probe 106 moves the first activation button 206a to the position shown in FIG. 100.

A reference marker 238 is shown in FIG. 100 to indicate the position of the bottom of the first activation button 206a when the first activation button 206a is in the end position. As shown in FIG. 100, the reference marker 236 and the reference marker 238 are a distance d apart from one another. Accordingly, when the operator moves the handheld ultrasound probe 106 upwardly from the position shown in FIG. 8 into contact with the first activation button 206a and the second activation button 206b and then causes the first activation button 206a and the second activation button 206b to move upwardly by the distance d, the scoop 214 moves from the default position shown in FIG. 9A to the end position shown in FIG. 9C. In one implementation, the distance d is 9/64^{th} of an inch.

When the operator moves the handheld ultrasound probe 106 away from the first activation button 206a and the second activation button 206b, the spring 240 decompresses and exerts a force on the first activation button 206a causing the first activation button 206a to move downwardly to the default position shown in FIG. 10A. As the first activation button 206a moves downwardly, the teeth 234 cause the gear 222 to rotate clockwise, which causes the scoop 214 to rotate clockwise until the scoop 214 returns to the default position shown in FIG. 9A.

FIG. 11 shows a portion of a container 250 according to one illustrated embodiment. The container 250 is similar in many relevant respects to the container 208. One difference is that the container 250 includes a spring 252 disposed between an upper portion 254 of the container 250 and a plunger 256. When the container 250 is initially filled with an ultrasound coupling medium 258, the spring 252 is compressed. Accordingly, the spring 252 exerts a force on the upper portion 254 and the plunger 256. The force exerted by the spring 252 causes the plunger 256 to be biased toward an elongated recess 260a of a scoop 260. Accordingly, the spring 252 may cause the plunger 256 to urge at least some of the ultrasound coupling medium 258 into the elongated recess 260a.

In one embodiment, the upper portion 254 is a removable lid. For example, side portions 264a and 264b of the container 250 may include recesses (not shown) into which opposing ends of the upper portion 254 may be inserted and removed to fasten the upper portion 254 to the container 250 and unfasten the upper portion 254 from the container 250, respectively. In one embodiment, the ultrasound coupling medium 258 is a gel contained in a bag 262 that is removably received in the container 250.

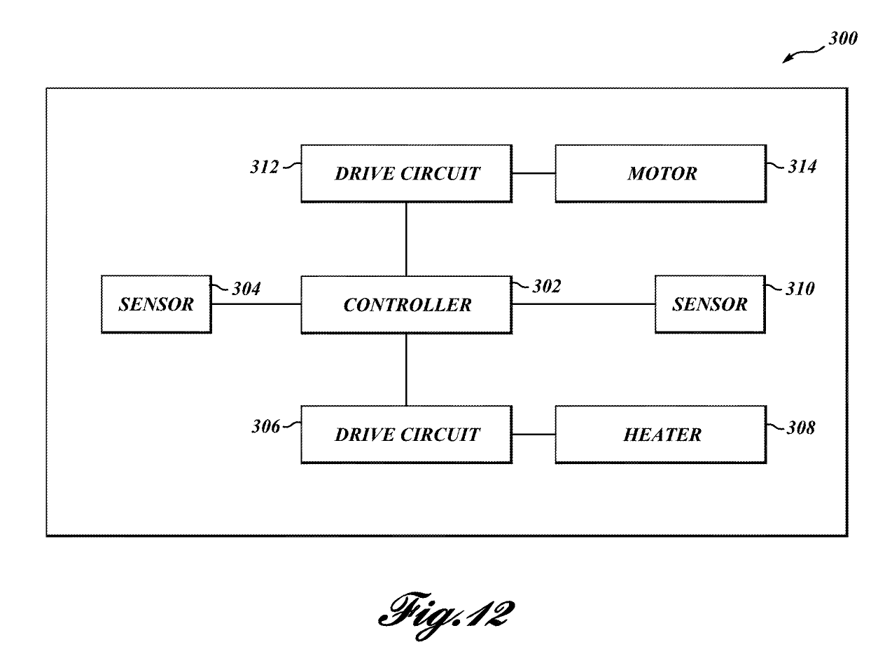

FIG. 12 shows a dispenser 300, according to one illustrated embodiment. The dispenser 300 includes a controller 302 electrically coupled to a temperature sensor 304 and a drive circuit 306, which is electrically coupled to a heater 308. The temperature sensor 304 is thermally coupled to the interior of a container (e.g., container 208) that may contain an ultrasound coupling medium.

The temperature sensor 304 provides a signal to the controller 302 indicating the temperature of the interior of the container.

The controller 302 stores a value corresponding to a desired temperature. The value corresponding to the desired temperature may be predetermined or may be set by an operator via an operator interface (not shown). For example, the operator interface may include an alphanumeric keypad that an operator can use to enter the value corresponding to the desired temperature.

When the signal provided by the temperature sensor 304 indicates that the temperature of the interior of a container (e.g., container 208) is a predetermined amount below the desired temperature, the controller 302 provides a signal to the drive circuit 306. In response, the drive circuit 306 causes a current to flow through the heater 308, which causes the heater 308 to produce heat. When the signal provided by the temperature sensor 304 indicates that the temperature of the interior of the container is the desired temperature, the controller 302 stops providing the signal to the drive circuit 306. In response, the drive circuit 306 stops causing the current to flow through the heater 308, which causes the heater 308 to stop actively producing heat.

In one implementation, the controller 302 is electrically coupled to a switch or sensor 310 and a drive circuit 312, which is electrically coupled to a motor 314. For example, the first activation button 206a and/or the second activation button 206b may be replaced with first and/or second proximity sensors that provide signals to the controller 302 when the handheld ultrasound probe 106 is in the position shown in FIG. 8. Alternatively, the first activation button 206a and the second activation button 206b may be replaced with first and second buttons each of which includes a switch or sensor 310 that provide a signal to the controller 302 when the buttons are depressed (e.g., by the first portion 106a and the second portion 106b of the handheld ultrasound probe 106, respectively).

When the switch or sensor 310 provides a signal to the controller 302, the controller 302 provides a signal to the drive circuit 312. In response, the drive circuit 312 outputs a signal that causes the motor 314 to rotate by a predetermined amount. For example, the motor 314 may be a stepper motor having a rotor coupled to a gear having threads that are dimensioned and positioned to engage the teeth 232 of the gear 222. When the switch or sensor 310 provides the signal to the controller 302, the controller 302 may provide a signal to the drive circuit 312 that causes the drive circuit 312 to control rotation of the rotor such that the scoop 214 rotates from the position shown in FIG. 9A to the position shown in FIG. 9C, and then rotate back to the position shown in FIG. 9A.

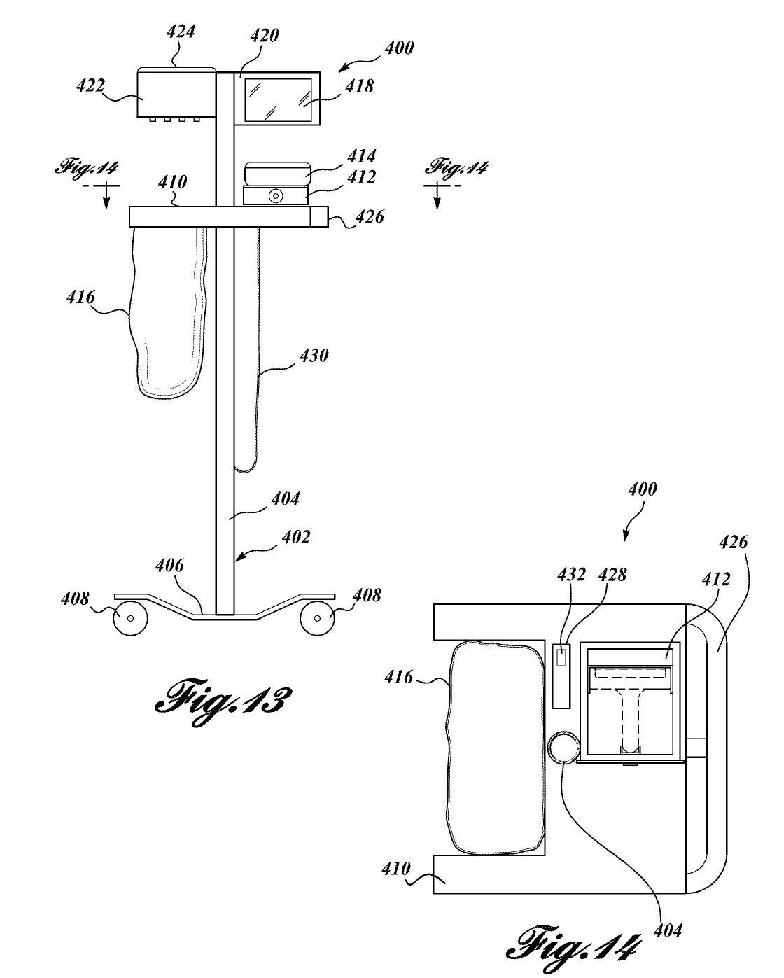

FIGS. 13 and 14 show a cart 400, according to one illustrated embodiment. The cart 400 includes a frame 402 comprised of a pole 404 that is coupled to a base 406. For example, an aperture having threads therein may be formed in a center portion of the base 406 and a bottom portion of the pole 404 may have complimentary threads formed thereon for attaching the pole 404 to the base 406 when the pole 404 is rotated clockwise.

A plurality of wheels 408 (e.g., 4) is rotatably mounted to the base 406 of the frame 402. For example, the wheels 408 may be of the swivel caster type, wherein each wheel 408 is rotatably mounted about a horizontal axle to which a fork is coupled. Each fork may be coupled to a swivel joint that rotates about a vertical axle that is inserted into an aperture formed in the base 406. Accordingly, the wheels 408 may enable an operator to move the cart 400 in any desired direction with very little effort.

A shelf 410 is coupled to the pole 404. For example, the pole 404 is inserted through an aperture formed in the shelf 410 and an L-shaped bracket is secured to the pole 404 and the shelf 410 using a plurality of bolts and nuts. In one embodiment, the shelf 410 is coupled to the pole 404 such that the height of the shelf 410 is adjustable.

A charging and sanitizing apparatus 412 (e.g., charging and sanitizing apparatus 100) is disposed on or in the shelf 410. In one embodiment, the charging and sanitizing apparatus 412 is coupled to the shelf 410 using at least one bolt and at least one nut to ensure that the charging and sanitizing apparatus 412 does not fall off the shelf 410, for example, while an operator is moving the cart 400.

A container 414 may be disposed on the shelf 410 or the charging and sanitizing apparatus 412. The container 414 may be sized and dimensioned to hold a supply of disposable alcohol or bleach wipes that can be used to sanitize parts of the cart 400, for example. A waste basket or bag 416 may be coupled to the shelf 410. For example, a plurality of clips (not shown) that can be removably secured to a plurality of portions of the bag 416 may be coupled to the shelf 410. The cart 400 may include one or more additional containers (not shown) that may be used to store one or more containers (e.g., containers 208) of an ultrasound coupling medium.

A display unit 418 is coupled to the pole 404. In one embodiment, the display unit 418 is of a type included in the console of the Benchmark Bladder System™ available from dBMEDx, Inc. The display unit 418 displays images based on data wirelessly received from an ultrasound probe (e.g., handheld ultrasound probe 106). For example, the display unit 418 may display a value indicating a measured volume of a bladder. In one embodiment, the display unit 418 includes a controller that is electrically coupled to a wireless receiver and a plurality of LEDs.

A mounting bracket 420 may be coupled to the display unit 418 and to the pole 404. In one embodiment, the mounting bracket 420 is coupled to the display unit 418 and the pole 404 using a plurality of bolts and nuts. In one embodiment, the mounting bracket 420 is coupled to the pole 404 such that the height of the display unit 418 is adjustable.

A dispenser 422 (e.g., dispenser 200) may be coupled to the pole 404 using a hanger 424. In one embodiment, the hanger 424 is coupled to the pole 404 and to the dispenser 422 using a plurality of bolts and nuts. In one embodiment, the hanger 424 is coupled to the pole 404 such that the height of the dispenser 422 is adjustable.

In one implementation, the dispenser 422 is detachably coupled to the pole 404. For example, the hanger 424 may be coupled to the pole 404 and may include a track into which an operator can insert a portion of the dispenser 422 to couple the dispenser 422 to the pole 404; the operator can remove the portion of the dispenser 422 to detach the dispenser 422 from the pole 404. An operator may remove the dispenser 422 from the hanger 424 so that the dispenser 422 can be placed on a patient to dispense an ultrasound coupling medium directly onto the skin of the patient, for example, onto the patient's abdominal region. In one implementation, at least part of the waste basket or bag 416 is positioned directly beneath at least part of the dispenser 422 such that, if the dispenser 422 is inadvertently activated, for example, any ultrasound compiling medium dispensed from the dispenser 422 falls into the waste basket or bag 416.

As shown in FIG. 14, a handle 426 is coupled to the shelf 410, for example, using a plurality of bolts and nuts. In one implementation, the handle 426 is integrally formed with the shelf 410. A towel (not shown) may be placed over the handle 426 such that a portion of the towel hangs between the shelf 410 and the handle 426, so that an operator can quickly access the towel during an ultrasound procedure, for example.

A power supply 428 is disposed in or on the shelf 410. In one embodiment, the power supply 428 is a model AHM85PS switching AC adapter available from Kowa Electronic Industries that can be provided with AC voltages ranging from about 80 V_{AC} to about 264 V_{AC}. In one implementation, the power supply 428 is coupled to the shelf 410 using at least one bolt and at least one nut to ensure that the power supply 428 does not fall off the shelf 410 while an operator is moving the cart 400, for example. The power supply 428 is electrically coupled to a power cord 430 (shown in FIG. 13), which may be electrically coupled to AC mains, for example. The power supply 428 also may be electrically coupled to the charging and sanitizing apparatus 412, the display unit 418, and the dispenser 422, for example, using a plurality of wires.

The power supply 428 may include a battery 432. For example, the power cord 430 may be plugged into an AC electrical outlet for a period of time sufficient to allow the battery 432 to become fully charged, to allow the charging and sanitizing apparatus 412 to fully charge the power storage device 124 of the handheld ultrasound probe 106, and to allow an ultrasound coupling medium contained in the dispenser 422 to be warmed to a desired temperature. After the power cord 430 is unplugged from the electrical outlet, the battery 432 may provide electrical power to the charging and sanitizing apparatus 412, the display unit 418, and/or the dispenser 422. Accordingly, the battery 432 of the power supply 428 may enable the charging and sanitizing apparatus 412, the display unit 418, and the dispenser 422 to be fully operational while the power supply 428 is not plugged into an electrical outlet.

The various embodiments described above can be combined to provide further embodiments. All of the U.S. patents, U.S. patent application publications, U.S. patent applications, International (PCT) patent applications referred to in this specification and/or listed in the Application Data Sheet, including but not limited to U.S. application Ser. No. 12/948,622, filed Nov. 17, 2010, International Application No. PCT/US11/26923, filed Mar. 2, 2011, U.S. Provisional Application No. 61/573,493, filed Sep. 6, 2011, U.S. Provisional Application No. 61/621,877, filed Apr. 9, 2012, U.S. Provisional Application No. 61/638,925, filed Apr. 26, 2012, U.S. Provisional Application No. 61/638,833, filed Apr. 26, 2012, U.S. Provisional Application No. 61/725,893, Nov. 13, 2012, U.S. application Ser. No. 13/800,993, Mar. 13, 2013, U.S. application Ser. No. 13/871,842, filed Apr. 26, 2013, U.S. application Ser. No. 13/871,835, filed Apr. 26, 2013, International Application No. PCT/US13/38505, filed Apr. 26, 2013, International Application No. PCT/US13/38479, filed Apr. 26, 2013, U.S. Provisional Application No. 61/876,018, filed Sep. 10, 2013, and International Application No. PCT/US13/68979, filed Nov. 7, 2013, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus for use with a handheld ultrasound probe, the handheld ultrasound probe which includes a power storage device, the apparatus comprising:
a holder that forms a compartment, the compartment sized and dimensioned to receive at least a portion of the handheld ultrasound probe therein;
a primary coil positioned with respect to the compartment to be proximate a secondary coil of the handheld ultrasound probe when the handheld ultrasound probe is positioned in the compartment;
a charging circuit electrically coupled to the primary coil to selectively provide an alternating current therethrough of sufficient strength to inductively couple the primary and the secondary coils when the handheld ultrasound probe is positioned in the compartment;
a plurality of light sources selectively operable to provide light at least from an ultraviolet band of an electromagnetic spectrum in at least part of the compartment to substantially illuminate the handheld ultrasound probe when the handheld ultrasound probe is positioned in the compartment; and
a drive circuit electrically coupled to drive the light sources.

2. The apparatus of claim 1, further comprising:
a cover, wherein at least one of the cover and the compartment is selectively movable between at least an open position in which the compartment is open to an exterior of the holder and a closed position in which the compartment is closed to the exterior of the holder.

3. The apparatus of claim 2 wherein the drive circuit drives the light sources to output ultraviolet light only when the cover or the compartment is in the closed position.

4. The apparatus of claim 1 wherein at least a first portion of the holder comprises at least one material that is transmissive of ultraviolet light.

5. The apparatus of claim 4 wherein the first portion of the holder is transmissive of visible light, and at least a second portion of the holder is formed from one or more materials that is nontransmissive of ultraviolet light.

6. The apparatus of claim 1 wherein the light sources include at least one ultraviolet light emitting diode.

7. The apparatus of claim 1, further comprising:
at least one adhesive that is transmissive of ultraviolet light and that secures the light sources to the holder.

8. The apparatus of claim 1 wherein at least one of the light sources is positioned adjacent a center portion of the primary coil and one or more one of the light sources is positioned adjacent an outer periphery of the primary coil.

9. The apparatus of claim 1 wherein the primary coil induces an electromagnetic field in the secondary coil of the ultrasound probe while the light sources output light.

10. A dispenser to dispense ultrasound coupling media, comprising:
a container having an interior that in use holds a supply of an ultrasound coupling medium; and
a dispensing mechanism having a scoop including at least an elongated recess, the elongated recess sized and dimensioned to hold a quantity of the ultrasound coupling medium, the scoop rotatably mounted with respect to the container to selectively move between at least a first position in which the elongated recess is in fluid communication with the interior of the container and to at least a second position in which the elongated recess is exposed to an exterior of the dispenser to dispense the quantity of the ultrasound coupling medium.

11. The dispenser of claim 10 wherein the scoop is an elongated cylindrical member with the elongated recess formed in a peripheral surface thereof.

12. The dispenser of claim 10 wherein the scoop is an elongated cylindrical member with the elongated recess having an arcuate profile.

13. The dispenser of claim 10 wherein the dispensing mechanism includes a plunger disposed in the container, the plunger movable to urge at least some of the ultrasound coupling medium into the elongated recess, and the dispensing mechanism also includes means for biasing the plunger toward the scoop.

14. The dispenser of claim 10 wherein the dispensing mechanism includes an actuator coupled to the scoop, wherein movement of the at least part of the actuator causes the scoop to rotate at least between the first and second positions.

15. The dispenser of claim 14 wherein
the actuator includes a first plurality of teeth,
the scoop is coupled to a second plurality of teeth that engage the first plurality of teeth, and
movement of the first plurality of teeth causes the scoop to rotate.

16. The dispenser of claim 10, further comprising:
a heater in thermal communication with the container or the interior of the container; and wherein the container includes at least one thermal insulator at least partially enclosing the interior thereof.

17. The dispenser of claim 10, further comprising:
a hanger to detachably couple the dispenser to a pole.

18. The dispenser of claim 10 wherein the ultrasound coupling media is a gel contained in a bag that is removably received in the container.

19. A cart, comprising:
a frame;
a plurality of wheels rotatably mounted to the frame;
a power supply carried by the frame;
a display unit carried by the frame, electrically coupled to the power supply;
an ultrasound probe holder that forms a compartment, the compartment sized and dimensioned to receive at least a portion of the handheld ultrasound probe therein;
a primary coil positioned with respect to the compartment to be proximate a secondary coil of the handheld ultrasound probe when the handheld ultrasound probe is positioned in the compartment;
a charging circuit electrically coupled to the power supply and to the primary coil to selectively provide an alternating current therethrough of sufficient strength to inductively couple the primary and the secondary coils when the handheld ultrasound probe is positioned in the compartment;
a plurality of light sources selectively operable to provide light at least from an ultraviolet band of an electromagnetic spectrum in at least part of the compartment to substantially illuminate the handheld ultrasound probe when the handheld ultrasound probe is positioned in the compartment; and
a drive circuit electrically coupled to drive the light sources.

20. The cart of claim 19 wherein the display unit displays images based on data wirelessly received from the ultrasound probe.

21. The cart of claim 19 wherein the ultrasound probe holder is formed from at least one material that is transmissive of ultraviolet light.

22. The cart of claim 19 wherein the primary coil induces an electromagnetic field in the secondary coil of the ultrasound probe while the light sources output light.

23. The cart of claim 19, further comprising:
a cover, wherein at least one of the cover and the compartment is selectively movable between at least an open position in which the compartment is open to an exterior of the holder and a closed position in which the compartment is closed to the exterior of the holder.

24. The cart of claim 23 wherein the drive circuit drives the light sources to output ultraviolet light only when the cover or the compartment is in the closed position.

25. The cart of claim 19 wherein at least a first portion of the holder comprises at least one material that is transmissive of ultraviolet light.

26. The cart of claim 19 wherein the power supply includes at least one battery.

27. The cart of claim 19, further comprising:
a dispenser including a container having an interior that in use holds a supply of an ultrasound coupling medium; and a dispensing mechanism having a scoop including at least an elongated recess, the elongated recess sized and dimensioned to hold a quantity of the ultrasound coupling medium, the scoop rotatably mounted with respect to the container to selectively move between at least a first position in which the elongated recess is in fluid communication with the interior of the container and to at least a second position in which the elongated recess is exposed to an exterior of the dispenser to dispense the quantity of the ultrasound coupling medium.

28. The cart of claim 27, further comprising:
a heater in thermal communication with the container or the interior of the container; and wherein the container includes at least one thermal insulator at least partially enclosing the interior thereof.

29. The cart of claim 27, further comprising:
a hanger to detachably couple the dispenser to the frame.
